(19) United States
(12) Patent Application Publication
(10) Pub. No.: US 2011/0208060 A1
(43) Pub. Date: Aug. 25, 2011

(54) Non-contact Biometric Monitor

(76) Inventors: Wayne C. Haase, Sterling, MA (US); Zachary S. Haase, Sterling, MA (US); Richard F. Young, Dover, MA (US); Philippe E. Monin, Glastonbury, CT (US); Colin W. McCarthy, Norwell, MA (US); F. William Sarles, Lexington, MA (US)

(21) Appl. No.: 13/034,394

(22) Filed: Feb. 24, 2011

Related U.S. Application Data
(60) Provisional application No. 61/338,738, filed on Feb. 24, 2010.

(51) Int. Cl. A61B 8/00

(52) U.S. Cl. 600/453

(57) ABSTRACT

A non-contact system utilizing an air-propagated ultrasound for monitoring biometric parameters is disclosed. A non-contact sensor transmits an ultrasonic wave toward a subject. The wave is reflected by the subject's skin surface back toward the sensor. Electronics in the sensor measure the small changes in displacement of the skin surface to derive a plurality of biometric parameters, including but not limited to respiration rate, heart rate, eye motion, and limb movement.

### CROSS REFERENCE TO RELATED APPLICATION

This application takes priority from and claims the benefit of Provisional Application Ser. No. 61/338,738 filed on Feb. 24, 2010 the contents of which are hereby incorporated by reference.

### BACKGROUND OF THE INVENTION

1. Field of the Invention

This invention relates generally to monitoring systems, and more particularly to portable, non-contact, non-invasive monitoring systems preferably for assessing a plurality of biometric information, including but not limited to respiratory parameters, cardiac parameters, ocular motion during sleep, and/or limb movement.

2. Description of the Related Art

The use of Doppler radar techniques to monitor subjects has been described in the literature. Some of the earliest work on the use of continuous wave (CW) Doppler radar to detect cardiopulmonary signals was done in the 1980s at the University of Illinois [2] using 10 GHz radar. In 2001, Lohman, et al., described signal processing techniques for a 2.4 GHz CW radar system [3]. More recently, several publications by Droitcour, et al., [4, 5] describe the use of 1.6 and 2.4 GHz CW radars to measure heart rate and respiration rate. These cited publications, as well as approximately ten others, are all based on Droitcour's Ph.D. Thesis [6], which provides a comprehensive discussion of the use of CW radar for measuring physiological parameters, including detailed analyses of system performance and limitations. The Droitcour Thesis discusses at considerable length the inability of a single-channel demodulator to provide a linear output for the phase-modulated echo from CW Doppler radar. The text concludes that a two-channel quadrature demodulator can be used with better results, simply by choosing the channel with the higher amplitude. However, the output of the quadrature demodulator is non-linear, which causes the shape and amplitude of the detected heart signals to vary depending on pulse amplitude and respiration.

In 2008, Jang, et al., described a system using 2.4 GHz CW radar [7] with a quadrature demodulator. The maximum distance of the radar source from a subject was 50 cm, at which point the signal-to-noise ratio (SNR) was 0 dB (noise power equals signal power). The cardiac waveforms described in the Jang, et al. publication vary in amplitude due to respiration, a result of the non-linear behavior of the quadrature demodulator.

U.S. Pat. No. 7,199,749[8], entitled “Radar detection device employing a scanning antenna system,” by Greneker, et al., describes a system developed at Georgia Tech Research Institute. This system uses a mechanical scanning system with a higher-frequency (10.525 GHz) radar in order to provide better spatial resolution and clutter reduction. Performance is comparable to the Droitcour system.

Table 1 shows typical performance characteristics of a radar-based system as described by Droitcour [6]. In this case, the antenna was approximately four inches square with a resulting beam whose electric field goes to zero at ninety degrees relative to the axis of primary transmission. Thus the 2.4 GHz radar signal that was transmitted covered a full hemisphere; that is, the beam was not like a focused flashlight, but more like an omni-directional flood lamp. A large percentage of the transmitted signal was reflected not from the subject but rather from stationary nearby objects producing “clutter.” The tests of the system were conducted in a radar-absorbing anechoic chamber and nearby chairs were covered in radar-absorbing materials to reduce clutter. The transmit/receive isolation (which is accomplished by an RF circulator) was 18 dB, meaning that a signal equal in amplitude to approximately 13% of the transmitted signal was added to the received signal. Thus it is clear that a large part of the receiver input signal was due to clutter, which is one of the reasons that the maximum range for the system was only one meter for cardiac measurements. Radar clutter cannot be reduced by increasing transmit power.

The Droitcour Thesis [6] noted that “The Doppler radar system was effective at measuring heart rate up to a range of one meter and measuring respiration up to a range of 1.5 meters. The current system was not accurate on a beat-to-beat basis [compared to the electrocardiogram], and this leaves much room for improvement in the signal processing.” One of the principal limiting factors for the SNR was phase noise in the 2.4 GHz Gunn oscillator. Because the reflected echo was mixed (multiplied) with the oscillator signal, the correlation decreased as the range increased (due to the time difference); the effect is called “range correlation” and causes SNR to decrease with increasing range, severely limiting the maximum range of the radar system. A commercial respiratory rate monitoring system using radar and based on the Droitcour Thesis is sold by Kai Sensors [9].

### SUMMARY OF THE INVENTION

The instant invention, as illustrated herein, is clearly not anticipated, rendered obvious or even present in any of the prior art mechanisms, either alone or in any combination thereof.

According to one aspect of the invention, a non-contact monitoring system preferably utilizing an air-propagated ultrasound for monitoring a plurality of biometric parameters is disclosed. One embodiment of the instant invention provides for a system comprising at least two ultrasonic transducers, wherein a first transducer is disposed to generate an ultrasonic beam to travel through the air to a subject being monitored by the system. Upon reaching the subject, the ultrasonic beam is reflected by the surface of the subject's skin back to the second transducer. Subsequently, a plurality of receiver electronics located within the transducers is able to detect variations in the returning echo to derive displacement, velocity, and/or acceleration of the subject's skin surface. Small displacements of the subject's skin surface are caused by breathing, heart motion, and pulsatile blood pressure. As a result, these small displacements are then detected by the system electronics and utilized to determine a plurality of physiological parameters such as heart rate, respiration rate, relative tidal volume, inspiration/expiration times, relative inspiration/expiration flow rates, eye motion (for identification of REM sleep), and limb movement.

Ideally, such systems may be used for diagnostics in a wide range of applications, including, but not limited to, monitoring subjects in critical-care environments, monitoring bed-ridden subjects in long-term care environments, monitoring neonates subject to sudden-infant-death syndrome (SIDS), and detecting symptoms of acute post-traumatic stress disorder (PTSD) or effects from traumatic brain injury (TBI) during sleep both in hospital and home environments [1]. The ability to assess biometric information in a non-contact manner minimizes adverse effects to the subject that may result from use of contacting or invasive devices. Such non-contact systems may also be used to assess potential hostile intent at security checkpoints by detecting unusual or changing biometric parameters.

Air-based ultrasound is capable of significantly improved performance over published radar approaches. The advantages of using ultrasound over radar in this application generally include higher spatial resolution, higher signal-to-noise, higher accuracy, smaller size, higher signal bandwidth, lower power operation, and improved subject safety.

Table 2 summarizes the differences between the radar and ultrasonic approaches, with a radar-based system operating at a frequency, f, of 2.4 GHz and an ultrasonic system at a frequency of 100 KHz. Even though the radar antenna and the ultrasonic transducer are of comparable diameter, D, the difference in propagation velocity, c, between the speed of light and the speed of sound produces a profound difference between the far-field beam patterns, which extend beyond the near field distance, L_{NF}. The angle of the main beam lobe, θ_{z}, and the angle at which the beam is at half-power, θ_{3dB}, for the two approaches are quite different. θ_{3dB} defined as the half-power beam width at a distance of 1 meter, is 3.5 meters (11.4 feet) for the 4-inch antenna of the Droitcour radar system but only 0.089 meters (3.5 inches) for an ultrasound system using the smaller 1.5-inch transducer shown in FIG. 10. While the radar antenna illuminates the whole room, including the entire subject and any others nearby, the beam from the ultrasonic sensor functions similarly to a flashlight, focusing only on a localized spot on a single subject. Therefore, an ultrasonic system may be designed to distinguish eye movement from respiratory or cardiac movement simply by aiming the transducer in the correct direction. Similarly, chest motion may be distinguished from jaw motion by proper aiming of the transducer; this technique is referred to as spatial filtering.

In addition to the lateral directions, spatial filtering may also apply to the direction of beam propagation; in this case, the controlling variable is the wavelength λ. Instead of transmitting a continuous wave (CW) as done for the Droitcour radar system, the ultrasonic system could be pulsed, allowing the same transducer to be used efficiently as a receiver. Echoes from close objects would arrive at the receiver earlier than those from distant objects; desired echoes could be separated from spurious echoes by range gating. For the expected range to the subject and ultrasonic frequency, this process would be relatively easy to accomplish for an ultrasonic system.

Similarly, such ultrasonic systems may be easily disposed to measure the distance to an object at a range of a few inches, primarily because the ultrasonic wavelength is 3.4 mm, or 0.135 inches. Therefore, spatial filtering in the beam propagation direction is relatively easy for radars designed to detect airplanes at ranges measured in miles, but not for the short ranges of a few meters. Measuring the distance to an object at a range of a few inches would be quite difficult for a 2.4 GHz radar system because its wavelength is 125 mm, or 4.9 inches.

In another embodiment of the instant invention, a single ultrasonic transducer is utilized in a pulsed mode wherein the transducer is disposed to provide both transmitting and receiving capabilities. Furthermore, the use of a pulsed mode allows the distance from the transducer to the subject to be measured. Thus, by using the echo from a specific time delay after the transmitted pulse, the distance at which the system is measuring may be controlled.

In yet another embodiment of the instant invention, a single ultrasonic transducer may be disposed to be constructed with a plurality of elements to form an array. The individual elements of the array may employ techniques that are well known in the art to form multiple ultrasonic beams. Therefore, depending on the specific circuitry of the transducer, these multiple beams may be separately steered toward different locations on the subject and can make simultaneous measurements from these multiple locations. A system designed in this manner can select the different locations on the subject for optimum detection of particular physiological parameters. For example, the optimum location for determining respiratory parameters may be on the chest or upper abdomen, whereas the optimum location for determining heart rate may be on the chest directly over the heart or on the neck near the carotid arteries. Similarly, detection of rapid-eye-movement (REM) sleep would be optimized by steering the beam toward the subject's eyes.

In a variation of the above embodiment, an array-based transducer would generate ultrasonic beams which would be steered in such a manner as to scan the subject—for example if the subject were lying on a bed—and thus determine both the body location and the specific body position. In this configuration, the system could adapt to a moving subject and maintain optimum measurement locations for the specific physiological parameters.

Furthermore, in terms of subject safety, the use of ultrasound provides a significant advantage over radar. Radar is subject to FCC regulations: transmission levels must be kept below power levels dictated by 47 CFR Part 15 for intentional radiators. Subject exposure to RF signals at radar frequencies (such as 2.4 GHz which is used both in the Droitcour system and in microwave ovens) must be limited, particularly for longer term monitoring. In addition, the proximity of a radar transmitter may create problems for other medical systems, such as heart pacemakers and other implanted devices. The use of a radar system for monitoring children may be considered highly problematic. Ultrasound, on the other hand, is completely safe, and is even used routinely to image babies in utero.

Additional features may be added to the base system, such as battery-power of the sensor and a wireless communication link to a laptop or computer network for additional analysis and data storage.

There has thus been outlined, rather broadly, the more-important features of a non-contact biometric monitor in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

Table 1 illustrates typical performance characteristics of a radar-based system for monitoring physiological parameters.

Table 2 illustrates and summarizes the differences between the radar and ultrasonic approaches, with a radar-based system operating at 2.4 GHz and an ultrasonic system at 100 KHz

FIG. 1 illustrates a diagrammatic perspective view of a monitoring system having a pair of ultrasonic transducers, wherein a first transducer is disposed for transmission and a second transducer is disposed for receiving echoes reflected from the subject.

FIG. 2 illustrates a block diagram for operation of a first embodiment of the monitoring system.

FIG. 3 illustrates a block diagram for operation of a second embodiment of the monitoring system.

FIG. 4 illustrates a displacement output of the monitoring system of FIG. 1 when the system is pointed at a subject's chest while the subject is breathing.

FIG. 5 illustrates a displacement output of the monitoring system of FIG. 1 when the system is pointed at a subject's neck to detect heart beats.

FIG. 6 illustrates an acceleration output of the monitoring system of FIG. 1 when the system pointed at a subject's neck to detect heart beats.

FIG. 7 illustrates a displacement output of the monitoring system of FIG. 1 when the system is pointed at a subject's face to detect eye motion.

FIG. 8 illustrates an acceleration output of the monitoring system of FIG. 1 when the system is pointed at a subject's face to detect eye motion.

FIG. 9 illustrates published electrocardiogram (EKG) waveforms along with carotid and ocular pressure pulses resulting from the heart beat.

FIG. 10 illustrates an electrostatic transducer developed by Polaroid Corporation for use in an auto-focus camera.

FIG. 11 illustrates a fan-beam configuration that may be generated by an array transducer.

FIG. 12 illustrates a modified beam configuration in contrast to FIG. 11 in which the elevation angle is fixed.

FIG. 13 illustrates the conversion of the ultrasonic transducer of FIG. 10 to an array-based device.

FIG. 14 illustrates a printed circuit board with vertical stripes that accomplishes the conversion of the ultrasonic transducer of FIG. 10 to an array-based device.

FIG. 15 illustrates a detailed shape of the beams of FIG. 12 that can be generated by the transducer of FIG. 10 coupled with the printed circuit board of FIG. 14.

FIG. 16 illustrates a printed circuit board with an interdigitated horizontal and vertical stripes composed of diamond-shaped elements.

### DETAILED DESCRIPTION OF THE PREFERRED EMBODIMENT

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

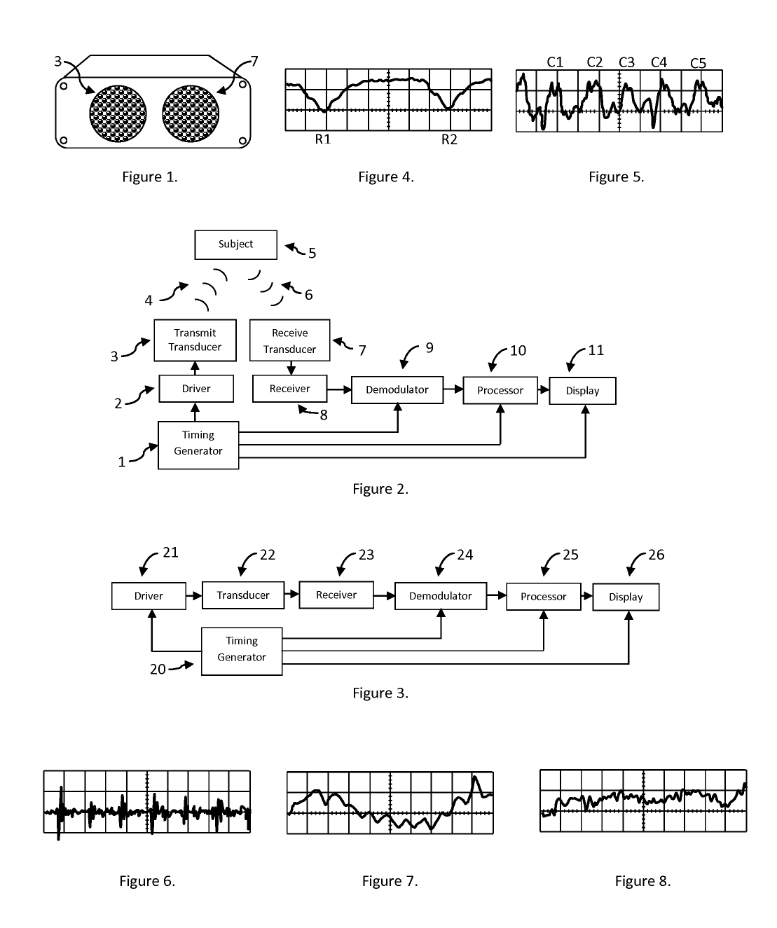

FIG. 1 illustrates one embodiment of the biometric monitoring system preferably comprising a pair of ultrasonic transducers, wherein the transducers may be made from piezo-ceramics (e.g., lead metaniobate or lead-zirconate-titanate), piezo-polymers (polyvinylidene-fluoride) or other materials. In one preferred configuration, the pair of ultrasonic transducers may be of an electrostatic type, as described by, Muggli, et al., in U.S. Pat. No. 4,081,626 [10]; by Paglia in U.S. Pat. No. 4,085,297 [11]; and/or by Kirby, et al., in U.S. Pat. No. 4,872,148 [12]. The unit shown in FIG. 1 uses two electrostatic transducers, which were developed by Polaroid® for an auto-focus camera, whereas FIG. 10 shows a single transducer.

FIG. 2 illustrates a block diagram of the first embodiment of the monitoring system in operation, wherein a timing generator 1 controls the system. The system further includes a driver 2 disposed to generate a continuous signal at a desired ultrasonic frequency which may be applied to a first transducer 3 disposed for transmission. In one embodiment, the ultrasonic frequency is typically in the range of 20 KHz to 100 KHz. The first transducer 3 emits an ultrasonic wave 4 that travels through the air to a subject 5 being monitored. Preferably the ultrasonic wave 4 will penetrate the subject's clothing and/or bedding material that may be in the path of the wave. In operation, the mismatch between the acoustic impedance of the air and the subject 5 causes ultrasonic energy to be reflected from the skin surface of the subject 5 allowing a subsequent ultrasonic wave 6 to return to a second transducer 7 for receiving the reflected ultrasonic wave. The transducers connect to a plurality of receiver electronics 8 disposed to convert the energy in the reflected ultrasonic wave 6 to a plurality of electrical signals forwarded to a demodulator 9. The demodulator 9 subsequently is disposed to translate the plurality of electrical signals into measurements of such parameters including, but not limited to as displacement, velocity, and acceleration of the subject's skin surface. Additional calculations may be done by a processor 10, which converts the displacement, velocity, or acceleration signals into physiological parameters such as breathing rate and heart rate. Calculated results are then passed to a display 11, which then displays the results either as waveforms or numeric values.

In a preferred embodiment, the demodulator 9 is disposed to determine the displacement of the skin surface of the subject 5 by comparing the phase of the reflected ultrasonic wave 6 to the phase of the initially transmitted ultrasonic wave 4 sent from the first transducer, wherein the phase difference indicates the displacement. Such phase comparison techniques are well known in the art and are commonly used in laser interferometry. Increased resolution of the phase difference may be realized by averaging techniques in which the phase difference is measured over multiple cycles of the ultrasonic signal. In a preferred embodiment, a signal proportional to the velocity of the skin surface of the subject 5 may be generated by differentiation with respect to time of the displacement signal. Similarly, a signal proportional to the acceleration of the skin surface of the subject 5 may be generated by differentiation with respect to time of the velocity signal.

As an example of the methods by which the processor 10 may calculate the various physiological parameters, the breathing rate may be calculated as follows: initially, the displacement signal may be passed through a bandpass filter to remove any dc or low frequency components and to remove high-frequency signals that would not be representative of breathing. Such high-frequency signals may be caused by cardiac motion but not by respiratory motion. The processor 10 may then measure the time between successive respiratory pulses to determine the breathing interval and may calculate the frequency of the respiratory pulses to determine breathing rate. Alternatively, the processor 10 may calculate the frequency components of the bandpass-filtered displacement signal by performing a fast Fourier transform (FFT) and may then estimate the breathing rate by selecting the signal with the highest amplitude. More-complex techniques, such a principal-component analysis may also be employed to estimate the breathing rate.

Furthermore, the processor 10 may also estimate the heart rate of a subject by similar filtering and calculation techniques as used for the breathing rate. Since the heart rate for a normal individual is generally much higher than the breathing rate, the cutoff frequencies for the bandpass filter would be generally set higher for heart rate calculations. Additionally, the processor 10 may also incorporate additional diagnostic capability, such as automatic alarms based on threshold limits for specific physiological parameters; this capability would be employed for monitoring systems intended to detect sleep apnea or Sudden Infant Death Syndrome (SIDS).

As an alternative, the demodulator 9 may employ standard techniques used in Doppler radar and sonar systems in which the received signal is mixed (multiplied) by two signals at the ultrasonic frequency and with ninety degrees relative phase to generate what are commonly called “quadrature” signals, or I (in-phase) and Q (quadrature) signals. Many of the references cited above which describe radar-based monitor systems (e.g., [6] and [7]) employ a quadrature demodulator.

FIG. 3 illustrates a block diagram of the second embodiment of the monitoring system, wherein in this embodiment a single ultrasonic transducer 22 is utilized instead of two transducers. The transducer 22 is disposed to operate in a pulsed mode, whereby a driver 21 initially applies a multi-cycle transmit burst controlled by a timing generator 20. After the completion of the transmit burst, an ultrasonic wave packet travels to the subject and is reflected from the skin surface as in the first embodiment. The received echo is then converted to an electrical signal by the same transducer 22, after which the signal processing and display of the physiological parameters proceeds as before.

An advantage of the second embodiment over the first embodiment is that the distance to the subject may be estimated by measuring the time delay between the transmission time of the ultrasonic wave packet and the received time of the echo. In addition, the received signal can be gated by the receiver, using such standard components as sample-hold circuits, to restrict the signal processing to only the echo occurring at a particular distance from the transducer 22. Such range-gating is commonly used in pulsed Doppler blood flow meters [13], whereby the flow measurements are restricted to the region of blood flow, and echoes from the blood vessel walls that occur before and after the flow echoes are eliminated. Such range-gating techniques provide the capability of spatial filtering and are relatively easy to accomplish in ultrasonic systems. In contrast, such range-gating techniques are relatively difficult to accomplish with radar-based systems due to the higher propagation velocity of radar signals compared to ultrasonic signals.

As was the case for the first embodiment, the transducers for the second embodiment can be of different types. In the preferred configuration, the transducers are electrostatic of the type developed by the Polaroid Corporation for an auto-focus camera, as shown in FIG. 10.

In yet another embodiment of the instant invention, a single ultrasonic transducer may be constructed with multiple elements to form an array. Beam shaping and beam steering techniques that are well known in the art may then be employed for multiple purposes. For example, a single, pencil-shaped beam may be generated for both transmit and receive functions, and used to scan the subject or to receive signals from only a specific location on the subject. In this general approach, the system could select the specific location on the subject that would maximize the ability to detect a physiological parameter. For example, the optimum location for determining respiratory parameters may be on the chest or upper abdomen, whereas the optimum location for determining heart rate may be on the chest directly over the heart or on the neck near the carotid arteries. Similarly, detection of rapid-eye-movement (REM) sleep would be optimized by steering the beam toward the subject's eyes. If the beam were used in such a manner as to scan the subject—for example if the subject were lying on a bed—the system could thus determine both the body location and the specific body position. In this way, the system would adapt to a moving subject and maintain optimum measurement locations for the specific physiological parameters.

An alternative approach to beam steering would be to transmit a horizontal, fan-shaped beam, and receive with a vertical, fan-shaped beam; this configuration based on two crossed linear arrays is often called a Mills Cross, named for the Australian radio telescope built by Bernard Mills. The intersection of the two fan beams would define the direction of a composite beam. The beam could be steered in elevation (up or down) by adjusting the timing of the transmit elements; the beam could be steered in azimuth (left or right) by adjusting phase delays on the receive elements. The Mills Cross configuration would allow for simpler transmit and receive circuitry compared to the more-general approach described above. A third possibility would be to transmit a horizontal, fan-shaped beam, and simultaneously receive on multiple, vertical fan-shaped beams.

FIG. 4 illustrates a displacement output signal from the demodulator 9 based on the embodiment in FIG. 1 with minimal band-pass filtering, as recorded on a digital oscilloscope. The vertical scale is 200 mV/division, and the horizontal scale is 1 second/division. In this embodiment, the ultrasonic frequency is approximately 100 KHz. Pulses R1 and R2 are representative of successive breaths as measured when the system is pointed at the chest of the subject 5 from a distance of approximately one meter with the subject 5 instructed to perform shallow breathing; the measurements were made through the subject's clothing, and as such, the low-amplitude signals on the waveform are due to cardiac motion. The waveform of FIG. 4 is downward during inhalation. Therefore, the breath rate is approximately ten breaths per minute, based on the time interval between pulses R1 and R2.

FIG. 5 illustrates a displacement output signal from demodulator 9 based on the embodiment of FIG. 1 wherein the system is pointed at the carotid artery of the subject 5. The vertical scale is 10 mV/division, and the horizontal scale is 0.5 seconds/division. Pulses identified as C1 through C5 are separate heart beats; the average heart rate of the subject 5 was approximately seventy beats per minute. The unusual negative pulse near the beginning of C1 and C4 is determined to be caused by premature ventricular contractions (PVCs) that the subject was experiencing. It is important to note that there is a small “dip” in the pulse near each peak, which is believed to be the dicrotic notch, the sudden drop in blood pressure after systolic contraction caused by a small reflux flow of blood back into the closing aortic valve and the coronary vessels.

FIG. 6 illustrates an acceleration output signal based on the embodiment of FIG. 1 wherein the system is pointed at the subject's neck. The vertical scale is 100 mV/division, and the horizontal scale is 0.5 seconds/division. In this embodiment, the shape of the individual beats is clearly distinguishable, with the waveform appearing more like an electrocardiogram (EKG).

Since acceleration is derived by a double differentiation with respect to time of the displacement waveform, it tends to emphasize the high-frequency components of the pulses. The effect can be understood more easily by noting that the second derivative of cos (ω) is −ω² cos (ωt); that is, the frequency components of the waveform itself are multiplied by frequency squared. Thus the acceleration (or possibly velocity, a single differentiation) output from the demodulator 9 might be better for determining heart rate or respiration rate (i.e., time-based parameters), whereas displacement may be better for amplitude-based parameters (such as maximum pressure or maximum volume).

FIG. 7 illustrates a displacement output signal from the demodulator 9 based on the embodiment of FIG. 1 wherein the system is pointed at the subject's eyes and the subject is instructed to keep their eyes closed, but to look left and right. The vertical scale is 50 mV/division, and the horizontal scale is 0.5 seconds/division. The low-frequency, large-amplitude component of the signal is due to the eye motion, and the smaller pulses on the waveform were attributed to changes in ocular pressure due to the subject's heart beats.

FIG. 8 illustrates an acceleration output signal from the demodulator 9 in a similar scenario; in this embodiment, the cardiac pulses are again readily apparent. The vertical scale is 20 mV/division, and the horizontal scale is 0.5 seconds/division.

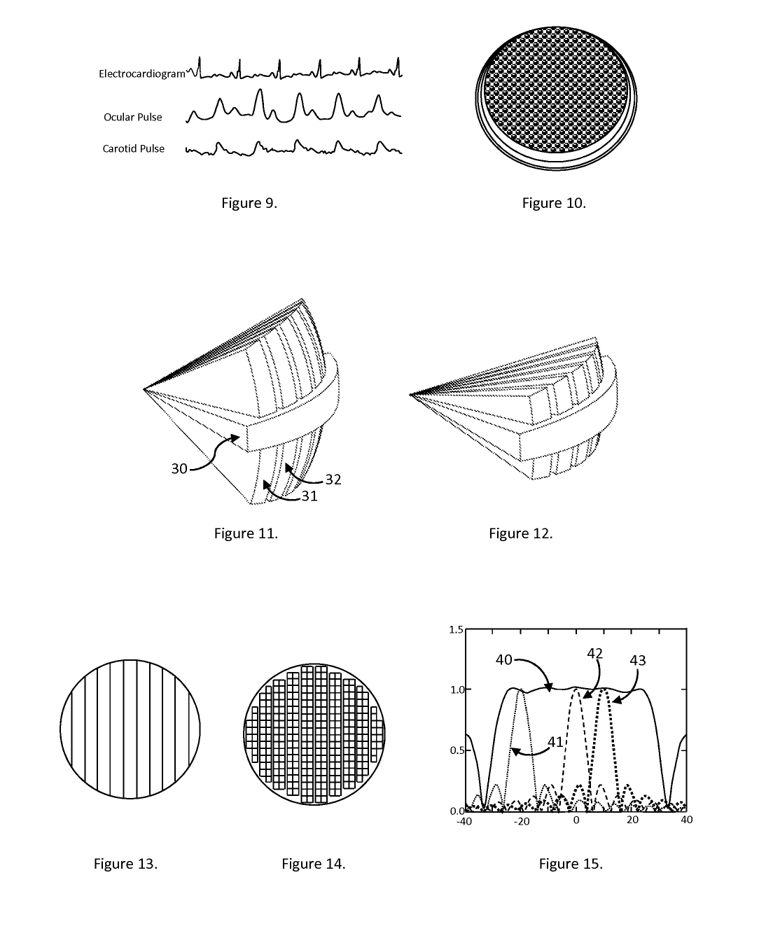

FIG. 9 illustrates a plurality of published waveforms of human ocular pulses [14]. The double-pulses that routinely occur in the ocular waveform are distinguished from the single pulses that occur in the carotid waveform. This effect is observed with the embodiment of FIG. 1, in which the ocular pulses (shown in FIG. 7) appear at twice the rate as the carotid pulses (shown in FIG. 5).

FIG. 11 illustrates the concept of the crossed beams. A horizontal fan beam 30 is generated by the array elements in transmit mode, and wherein a pair of vertical fan beams 30, and 31, among others, are used in receive mode. The intersection of the beams would define the region from which the data would be collected. The elevation angle of transmit fan-beam 30 could be varied in order to collect data from other regions.

If the elevation angle of the transmit fan-beam 30, could be held constant, the beams could be simplified as shown in FIG. 12. In this case, the elevation angle of the receive fan beams has been reduced to match the elevation angle of the transmit beam. Thus the receive beams would be converted to pencil-beams, rather than fan-beams. The configuration of FIG. 12 would allow the monitoring system to simultaneously collect information from different locations on the subject to optimize the measurements of the different physiological parameters. This approach would be similar to that used in underwater diver-detection sonar systems. For transmit, these systems typically use a broad beam that insonifies the entire region to be monitored, such as a harbor; for receive, they use an array transducer which generates multiple parallel beams to allow simultaneous monitoring in multiple directions.

Applying the approaches illustrated in FIGS. 11 and 12 to a biometric monitor, an array transducer may transmit a broad beam that covered the chest, neck, and head of the subject, and operate with separate receive beams that could be independently steered to specific locations on the subject. An additional receive beam could be used to scan the subject to detect body position and to determine where to aim the other beams for optimum signal detection. Thus beam steering would accomplish spatial filtering and the system could separate eye motion from limb, chest, or jaw motion. Frequency filtering could be added to further separate cardiac from respiratory motion for situations in which both signals were present, such as for the chest.

FIG. 13 illustrates how the circular Polaroid® transducer may be converted to an array transducer. In this particular embodiment, the backing plate of the transducer would be removed and replaced with a planar array of vertical stripes. Such a pattern can be readily fabricated using conventional printed circuit board (PCB) technology. Standoff spacers are generated by appropriate use of thin sections of solder mask. Connections to each of the stripes would be accomplished with feed-through holes to the opposite side of the PCB.

FIG. 14 illustrates a PCB with ten vertical stripes as shown by FIG. 13. Each of the elements in the ten stripes would be connected together electrically. The resulting beam patterns for transmit and receive would be similar to FIG. 12.

FIG. 15 illustrates detailed beam patterns that may be generated by the transducer that utilizes the PCB of FIG. 14. The vertical scale represents normalized amplitude and the horizontal scale represents angle in degrees. A fan-beam 40 is used for transmission, and a plurality of pencil-beams 41, 42, and 43, are used for receiving transmissions.

The generation of the various fan-beams and pencil beams may be accomplished by techniques described in U.S. Pat. Nos. 4,431,936 and 4,519,260 by Fu and Gerzberg [15, 16]. The transmit fan-beam would be generated by adjusting the amplitude and phase of the vertical stripes of FIG. 14. In particular, the center stripes would be at full amplitude, and succeeding stripes would be at lower amplitude and inverted in phase relative to the preceding adjacent stripe. In this manner, only two transmit phases (0° and 180°) would be required.

The receive pencil-beams of FIG. 12 may be generated in several ways. One would be to adjust the phase of the individual receiver circuits, prior to summation into a single composite signal. A preferred way would be to use the approach of digital beam-formers, in which the signal from each of the stripes is separately digitized by an analog-to-digital converter (ADC). By combining the ADC outputs from different sample times, the separate receive beams are generated simultaneously.

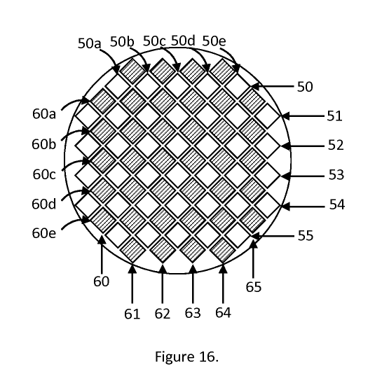

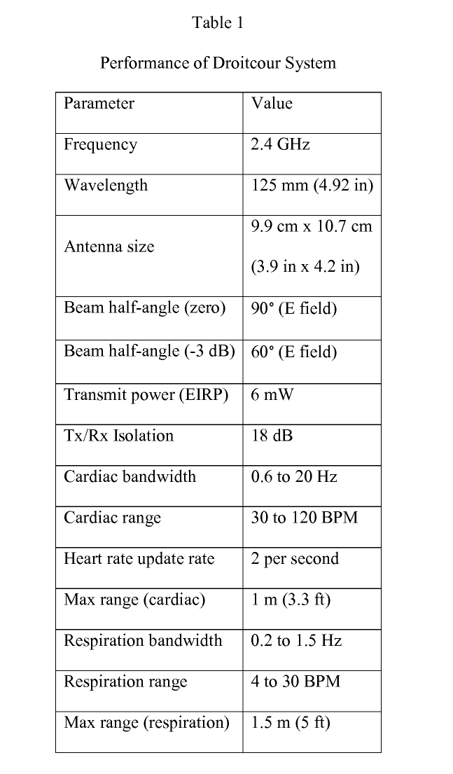

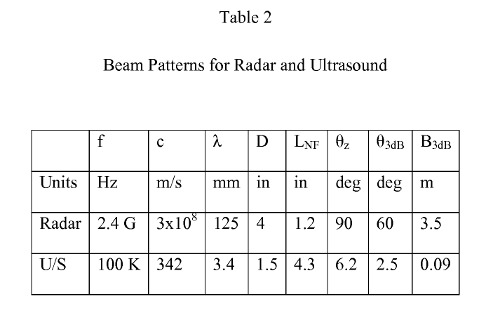

FIG. 16 illustrates a PCB pattern capable of generating the beam patterns of FIG. 11. The pattern is a two-dimensional array of diamond-shaped elements, which are grouped as interdigitated horizontal rows and vertical columns. For example, a horizontal row 50 is comprised of five elements labeled 50a through 50e. A second horizontal row 51 is below row 50 and is comprised of seven elements (not individually labeled in the drawing). Furthermore, a plurality of horizontal rows 52 through 54 is likewise comprised of seven elements. The bottom horizontal row 55 is comprised of five elements. The remaining elements in FIG. 16 comprise six vertical columns, labeled 60 through 65. The vertical column 60 is comprised of five diamond-shaped elements labeled 60a through 60e. Vertical columns 61 through 64 are comprised of seven elements (not individually labeled in the drawing). Column 65 is comprised of five elements. The figure thus illustrates an efficient technique for interleaving a horizontal and a vertical array.

The grouping of the elements of FIG. 16 provides an efficient method to distribute the elements in the outline of a circular transducer, as shown in FIG. 10. The separation of the elements into rows and columns further simplifies the electronics required to generate the beam pattern of FIG. 11. In particular, the horizontal rows can be used for the transmit function of the transducer, and the vertical columns can be used for the receive function. The beam shaping and steering techniques associated with FIG. 15 and discussed above would be used to generate the specific beam patterns.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

### REFERENCES

1. Elsevier Health Sciences. “Promising Treatment for Post Traumatic Stress Disorder Sleep Disturbances.” Science Daily, 19 Apr. 2007. http://www.sciencedaily.com/releases/2007/04/070418091940.htm
2. “Remote Sensing of Body Signs and Signatures,” J. Lin and K. Chan, Department of Bioengineering, University of Illinois at Chicago, Contract No N00014-82-C-0659, Naval Medical Research and Development Command, National Naval Medical Center, Bethesda, Md. 20814, September, 1985
3. “A Digital Signal Processor for Doppler Radar Sensing of Vital Signs,” B. Lohman, O. Boric-Lubecke, V. Lubecke, P. Ong, M. Sondhi, 23^{rd} International Conference of the IEEE Engineering in Medicine and Biology, Istanbul, Turkey, Oct. 25-28, 2001
4. “Range Correlation and I/Q Performance Benefits in Single-Chip Silicon Doppler Radars for Noncontact Cardiopulmonary Monitoring, A. Droitcour, O. Boric-Lubecke, V. Lubecke, J. Lin, G. Kovacs, IEEE Transactions on Microwave Theory and Techniques, Vol 53, No 3, March 2004
5. “Range Correlation and I/Q Performance Benefits in Single Chip Silicon Doppler Radars for Non-Contact Cardiopulmonary Monitoring,” A. Droitcour, O. Boric-Lubecke, V. M. Lubecke, J. Lin and G. T. Kovacs, IEEE Transactions on Microwave Theory Tech., Vol. 52, No. 3, pp. 838-848, March 2004
6. “Non-Contact Measurement of Heart and Respiration Rates with a Single-Chip Microwave Doppler Radar,” A. Droitcour, Ph.D. Thesis, Stanford University, Stanford, Calif., June 2006, 428 pages
7. “Wireless Bio-Radar Sensor for Heartbeat and Respiration,” B. Jang, S. Wi, J. Yook, M. Lee, K. Lee, Progress in Electromagnetics Research, C. Vol 5, 2008
8. “Hand-held radar device detects breathing, heartbeats,” Design News, Boston, Jan. 19, 1998, Vol 53, No. 2, p 36
9. Kai Sensors, 3465 Waialae Avenue, Suite 370, Honolulu, Hi. 96816 http://www.kaisensors.com/index.php
10. U.S. Pat. No. 4,081,626, “Electrostatic Transducer having Narrowed Directional Characteristic,” Muggli, J., and Specht, M. G., and assigned to Polaroid Corporation, Cambridge, Mass.
11. U.S. Pat. No. 4,085,297, “Spring Force Biasing Means for Electroacoustical Transducer Components,” Paglia, R., assigned to Polaroid Corporation, Cambridge, Mass.
12. U.S. Pat. No. 4,872,148, “Ultrasonic Transducer for use in a Corrosive/Abrasive Environment,” Kirby, J. P., and Paglia, R., assigned to Polaroid Corporation, Cambridge, Mass.
13. “A Directional Pulsed-Doppler Ultrasonic Blood Flowmeter,” W. C. Haase, Ph.D. Thesis, Stanford University, Stanford, Calif., April, 1978.
14. “Noncontact detection of ocular pulse—correlation with carotid stenosis,” J. Zuckerman, K. Taylor, H. Grossman, Invest. Ophthalmology Visual Sci, November, 1977, pp 1018-1024
15. “Transducer structure for generating uniform and focused ultrasonic beams and applications thereof,” C. Fu, L. Gerzberg, U.S. Pat. No. 4,431,936, Feb. 14, 1984
16. “Ultrasonic transducers and applications thereof,” C. Fu, L. Gerzberg, U.S. Pat. No. 4,519,260, May 28, 1985

We claim:

1. An apparatus for non-contact monitoring of a plurality of physiological parameters of an individual, the apparatus comprising:
a. a first ultrasonic transducer;
b. a second ultrasonic transducer; and
c. a plurality of signal-processing electronics disposed to derive the physiological parameters of the individual being monitored based on the reflected ultrasonic wave.

2. The apparatus of claim 1 wherein the first ultrasonic transducer is an ultrasonic transmitter disposed to transmit ultrasonic wave toward the individual being monitored and the second ultrasonic transducer is an ultrasonic receiver disposed to receive ultrasonic wave transmissions from the individual being monitored; wherein the ultrasonic wave transmission is reflected from an area of the skin surface of the individual being monitored.

3. The apparatus of claim 1 wherein the plurality of signal-processing electronics is disposed to derive a plurality of physiological parameters based on measurements of displacement, velocity, and/or acceleration of the individual's skin surface.

4. The apparatus of claim 1 wherein the ultrasonic transmitter and the ultrasonic receiver are combined as a single ultrasonic transducer.

5. The apparatus of claim 4 wherein the plurality of signal-processing electronics is disposed to derive the distance between the ultrasonic transducer and the individual.

6. The apparatus of claim 5 wherein the plurality of signal-processing electronics is disposed to derive a plurality of physiological parameters based on signals from a specific range of distances from the ultrasonic transducer.

7. The apparatus of claim 4 wherein the ultrasonic transducer comprises multiple elements arranged in the form of an array.

8. The apparatus of claim 7 wherein the array is disposed to comprise a linear array of elements.

9. The apparatus of claim 7 wherein the array is disposed to comprise a two-dimensional array of elements.

10. The apparatus of claim 7 wherein the ultrasonic transmit wave and the ultrasonic receive wave are disposed to be steered in different directions in azimuth and an elevation relative to the axis of the transducer.

11. The apparatus of claim 7 wherein the ultrasonic transmit beam and the ultrasonic receive beam patterns are disposed to be narrow in both azimuth and elevation.

12. The apparatus of claim 7 wherein:
a. the ultrasonic transmit beam is disposed to be narrow in one dimension and wide in the orthogonal direction in a fan shape, and
b. the ultrasonic receive beam is a single beam is disposed to be narrow in both dimensions in a pencil shape.

13. The apparatus of claim 7 wherein
c. the ultrasonic transmit beam is a single beam disposed to be narrow in one dimension and wide in the orthogonal direction in a fan shape; and
d. the receive beam is a single beam disposed to be narrow in one dimension and wide in the orthogonal direction in a fan shape; and
e. the receive fan shape is orthogonal to the transmit fan shape.

14. The apparatus of claim 7 wherein:
f. the ultrasonic receive beam is comprised of multiple beams that are substantially non-overlapping; and
g. the plurality of signals are received simultaneously by the receive beams from different locations on the individual.

15. The apparatus of claim 14 wherein the receive beams are separately steered to locations on the individual that are optimized for measurement of the particular physiological parameters.

16. The apparatus of claim 7 wherein:
h. the ultrasonic transducer is comprised of a flexible, metalized film and a printed circuit board; and
i. the array of multiple elements are defined by metal patterns on the printed circuit board.

17. The apparatus of claim 7 wherein the array elements are arranged and interconnected in the form of parallel stripes.

18. The apparatus of claim 7 wherein the array elements are arranged in an interdigitated pattern of horizontal rows and vertical columns.

19. The apparatus of claim 7 wherein the array elements in the rows and columns are generally of a diamond shape.

20. A method for non-contact monitoring a plurality of physiological parameters of an individual utilizing the apparatus of claim 1, the method comprising the steps of:
j. transmitting an ultrasonic wave toward the individual being monitored;
k. receiving the reflected ultrasonic wave from the individual being monitored; and
l. processing the received ultrasonic signal to derive the physiological parameters of the individual being monitored.
